(19) United States
(12) Patent Application Publication
(10) Pub. No.: US 2011/0008801 A1
(43) Pub. Date: Jan. 13, 2011

(54) METHODS FOR MEASURING CELL-CELL OR CELL-MATRIX ADHESIVE FORCES AND COMPOUNDS FOR DISRUPTING ADHESIVE FORCES IN BIOLOGICAL SYSTEMS

(75) Inventors: Terri A. Camesano, Newton, MA (US); Yatao Liu, Worcester, MA (US); Amparo Gallardo-Moreno, Badajoz (ES)

Correspondence Address: BURNS & LEVINSON, LLP, 125 SUMMER STREET, BOSTON, MA, 02110, US

(73) Assignee: Worcester Polytechnic Institute, Worcester, MA (US)

(21) Appl. No.: 12/293,728

(22) PCT Filed: Mar. 20, 2007
(86) PCT No.: PCT/US07/64446

§ 371 Date: Sep. 20, 2010

Related U.S. Application Data
(60) Provisional application No. 60/783,968, filed on Mar. 20, 2006.
(60) Provisional application No. 60/784,035, filed on Mar. 20, 2006.

(51) Int. Cl. G01N 33/53; C12N 1/20; C12Q 1/02

(52) U.S. Cl. 435/7.21; 435/252.1; 435/252.8; 435/29; 435/7.32; 435/7.37; 435/325

(57) ABSTRACT

The invention provides an atomic force microscope-based bioassay, assisted by thermodynamic characterizations to quickly and accurately screen for compounds that disrupt cell-cell or cell-substrate interactions.

### BACKGROUND OF THE INVENTION

Atomic Force Microscopy (AFM) high resolution imaging and force measurements have been widely used to characterize bacterial cells. Earlier researchers used glutaraldehyde to immobilize bacterial cells onto an AFM tip, but this causes protein cross-linking and makes the cell surface artificially rigid. The force measurements obtained using this method were therefore not representative of true cellular interactions. An additional difficulty to overcome is related to reproducibly attaching a certain number of cells to the AFM tip. Considering the curvature of the AFM tip, only a few cells of the size of bacterial cells should be attached and one cell is highly preferred since many applications require the measurements of single cell interaction forces. When an excess of cells are attached, the top layer cells are usually loosely attached, hence they may detach when force measurements are conducted in liquid. The detached cells can then be detected on the substrate by the AFM probe, leading to erroneous force measurements. Further, the number of attached cells determines the contact area with the substrate. Although it is known that the value of the adhesion forces is directly related to the contact area, the prior art in this area could not control how many cells will be attached onto the AFM tip, especially at the crucial location of the tip apex.

Some previous researchers have chosen not to use whole cells, but to use analogues of receptors and ligands. For example Srikanth et. al. (Langmuir 2003) probed the interactions between cholera toxin B oligomer and its receptor ganglioside GMI. A drawback of using isolated receptors and ligands is that the conformation or orientation of these molecules is different from the native structure of real receptors existing on cell surfaces.

Interfacial free energy analysis derived from contact angle measurements has also been widely used by numerous researchers. This method is quite successful to explain the transport of bacterial cells in groundwater or the subsurface soil environment, and the adhesion ability of bacterial cells to environmental surfaces. There are only a few examples of the use of this method for cells, besides the environmental studies. For example, Mabboux et. al. (Colloids and Surfaces B: Biointerfaces 2004) investigated the surface free energy of bacterial cells and saliva-coated dental implant materials (pure titanium grade and its alloy). They related the surface free energy with retention of bacteria based on in vitro retention experiments. The two surfaces in this system are one biological surface (bacterium) and one inert surface (the dental implant materials). Also the prior art did not consider the system interfacial free energy. They only considered one component of the system interfacial free energy, i.e. the surface free energy of the substrate and ignored the other two components, those arising from the bacterial cells and the medium. In another biological study, Braga et. al. (Pharmacological Research 1995) alluded that interfacial free energy was correlated with the adhesion of Staphylococcus aureus to epithelial cells under the influence of subinhibitory concentrations of brodimoprim. However, they only measured the water contact angles on bacterial cells, which is actually only a small part of measuring and calculating a system interfacial free energy. They also ignored the energy contributions due to the epithelial cells and the medium.

In brief, the interfacial free energy method has been successfully applied in environmental studies, which only comprises one live cell and the inert substrate. The method described herein is brand new in the biological and biomedical fields, where systems consist of two living cells. Specifically, the current method determines the interfacial free energy of E. coli bacterial cells and uroepithelial cells under the influence of cranberry juice and its selected components. The method of the invention can be used to examine other biological and biomedical problems, such as various other baeterial mammalian cell pairs involved in infections.

### SUMMARY OF THE INVENTION

The invention includes, “Atomic force microscope-based bioassay, assisted by thermodynamic characterizations to quickly and accurately screen the active component(s) in American red cranberry (Vaccinium macrocarpon Ait., family Ericaceae)”, by
(1) assaying the active components in American red cranberry (Vaccinium macrocarpon Ait., family Ericaceae) and
(2) determining the optimal dose (either concentration or dose per cellular units) and the optimal time needed for the cranberry juice and the active components to act.

More generally, the invention provides a method for functionalizing an atomic force microscope (AFM) tip by attaching a single cell to the top of the tip. To attach a single cell to a tip, the tip is coated with positively charged polymer molecules, and then the polymer coated tip is brought into contact with a cell suspension. The attachment of a single cell to an AFM tip can be verified by one or more of the following methods: (1) imaging by scanning electron microscopy, (2) determining the resonance frequency shift, (3) measuring the difference in the characteristic force spectra.

The types of AFM tips that can be functionalized with a single cell, include but are not limited to, silicon tips, silicon nitride tips and gold-coated tips. Positively charged polymer molecules may include poly-L-Lysine or poly-(ethyleneimide).

The types of cells that can be attached to an AFM tip, include but are not limited to bacterial cells, such as Escherichia coli, Helicobacter pylori and Porphyromonas gingivalis.

Another embodiment of the invention includes a method for measuring adhesion forces between two cells or between a cell and a substrate by determining the system interfacial free energy by measuring the surface free energy of a first cell; measuring the surface free energy of a second cell; and measuring the surface free energy of a medium. Forces can be measured between different types of cells including bacterial cells and mammalian cells, including but not limited to epithelial cells and erythrocytes. Substrates may include gastric mucous or protein-coated surfaces such as, periodontal tissue or teeth coated with collagen (including type I collagen), fibrinogen, or serum.

In some embodiments the effects of chemical compounds on the adhesion forces between cells can be investigated. One such example is to measure the effects of cranberry juice or isolated components of cranberry juice. The methods can be performed to quickly and efficiently screen for compounds and the appropriate concentrations of compounds that can disrupt cell-cell or cell-matrix interactions.

### BRIEF DESCRIPTION OF THE DRAWINGS

FIG. 1A is a photograph of E. coli HB101 grown in TSB;

FIG. 1B is a photograph of E. coli HB101 grown in TSB supplemented with 10% neutralized CJC;

FIG. 2 is a bar graph of the adhesion force between individual bacterial cells of E. coli HB101pDC1 (expresses P fimbriae) and uroepithelial cells, probed in phosphate buffered saline (PBS), or in buffer supplemented with CJC at concentrations of 2.5, 5.0 or 10.0%;

FIG. 3 is a SEM imaging of one bacterium functionalized AFM tip;

FIG. 4 is a graph of the resonance frequency tune given by AFM;

FIG. 5 is a graph of the comparison between interaction forces of bacteria coated AFM tip and bare tip;

FIG. 6A is a graph of the contact angle as a function of drying time for P-fimbriated E. coli:

FIG. 6B is a graph of the contact angle as a function of drying time for Uroepithelial cells;

FIG. 7 is a diagram of the method used to investigate surface free energy;

FIG. 8 is a graph of the interfacial free energy between E. coli and uroepithelial cells;

FIG. 9 is a graph of the interfacial free energy between E. coli and uroepithelial cells with different cranberry juice treatments;

FIG. 10 is a diagram of the nanoscale tool, atomic force microscope;

FIG. 11 is a graph of the measure of adhesion force as a function of the distance of the tip of the AFM from the substrate;

FIG. 12A is a bar graph of the interaction forces between E. coli HB101pDC1 and Uroepithelial cells;

FIG. 12B is a bar graph of the interaction forces between E. coli HB101 and Uroepithelial cells;

FIG. 13 is a graph of the interfacial free energy caused by HB101 vs. HB101pDC1;

FIG. 14 is a table of the results for contact angles and surface free energy components of HB101pDC1 before and after cranberry treatment;

FIG. 15 is a table of the results for contact angles and surface free energy components of HB101 after a 3-hour incubation with or without cranberry;

FIG. 16 is a table of the results for contact angles and surface free energy components of uroepithelial cells after a 3-hour incubation with or without cranberry;

FIG. 17A is a bar graph of the effects of cranberry juice on the conformation of P-fimbriae (length) for HB101pDC1;

FIG. 17B is a bar graph of the effects of cranberry juice on the conformation of P-fimbriae (length) for HB101; and

FIG. 17C is a bar graph of the effects of cranberry juice on P-fimbriae (mass of the biomacromolecules per cell surface area.

### DETAILED DESCRIPTION OF THE INVENTION

As the second most common type of infection, urinary tract infections (UTIs) are especially a concern for women, ⅓ of whom will have at least one UTI in their lifetime (Sobel, 2000), leading to the infection of 11.3 million women per year in the U.S. alone (Foxman 2000). Elderly women and children are also extremely prone to UTIs, with some women over 65 experiencing at least one UTI per year (Boscia, Kobasa et al. 1986; Abrutyn, Mossey et al. 1991). The prior art especially the in vivo clinical studies has conclusively proved that cranberry juice can prevent UTIs by decreasing bacterial adhesion onto uroepithelial cells. Some recent suggests that cranberries can be a possible treatment for UTIs, although more clinical studies need to be done. The clinical studies are difficult, expensive, and time-consuming. In order to optimize such studies, one must determine the appropriate dose and concentration of cranberry to administer, as well as the best individual conditions and sample size. Indeed, the studies within the prior art differ from each other regarding the dose and effects. Some researchers found that cranberry tablets could not change urinary pH, or reducing bacterial counts, urinary WBC counts, or UTIs in individuals with neurogenic bladders. While other studies have conclusively demonstrated that WBC counts and bacterial counts were reduced by cranberry juice and table consumption, as well as preliminary evidence that bacterial counts are reduced after consumption of sweetened dried cranberries. It is not feasible to test all of the possible doses and combinations of active ingredients in cranberries at a clinical stage at this time.

Therefore, other previous work has focused on in vitro studies, which can eliminate some of the uncertain and uncontrolled factors to better determine the mechanisms of cranberries actions against E. coli and uroepithelial cells. In vitro bacterial retention experiments are the most common. Experiments either focus on bacterial retention to epithelial cells or bacterial retention to polyethylene beads that are functionalized with epithelial cell receptor analogues. It is difficult to conduct the experiments reproducibly since laboratory difficulties such as removing the loosely attached bacterial cells from epithelial cells do not always yield consistent results. Many trials are required to obtain statistically meaningful data, consuming much time and labor. The intensive and manual nature of these experiments also opens such assays to the high possibility of operator error. This method is not suitable to quickly screen active component(s) in cranberry juice formulations.

Our invention, the AFM-based bioassay, assisted by thermodynamic characterizations, can overcome the aforementioned drawbacks and provide an accurate and fast method to research the active component(s), allowing for further study of the mechanisms of cranberry's effects against E. coli and uroepithelial cells. This optimization will allow us to better utilize such component(s) to increase the health and wellness of the population.

### Bacterial Adhesion

The adherence of bacteria to cells or tissues in the body is the propagating step in infections. Bacterial surfaces contain several types of molecules that help them attach to cells, such as proteinaceous fimbriae or pili, flagella, lipopolysaccharides, and capsular polysaccharide molecules. When the bacterial structures find their complementary receptors on mammalian cells, the two bind tightly. In the case of urinary tract infections, fimbriae expressed by Escherichia coli (E. coli) must bind to receptors on uroepithelial cells. A similar mechanism exists in gastric ulcers. In the case of a Helicobacter pylori infection, which can lead to the development of a gastroduodenal ulcer, bacteria must attach to human gastric mucosal cells for the infection to develop. A third bacterial infection that develops following adhesion of bacteria is related to periodontitis, an inflammatory disorder of tooth-supporting tissues. Gram-negative bacteria, such as Porphyromonas gingivalis can colonize teeth, gingival epithelial cells, and red blood cells, or interact with other oral bacteria and proteins in the mouth through receptors on their surfaces (1).

Compounds that prevent adhesion of bacteria to mammalian cells represent an alternative therapy to the use of antibiotics, since the anti-adhesion molecules do not kill or impair the growth of the bacteria, yet they are able to prevent the infection from developing. Cranberry compounds have been implicated in preventing the bacterial adhesion process, thus presenting a complementary or alternative methodology to prevent urinary tract infections (2, 3), H. pylori infections (4, 5), and periodontitis (1).

### Cranberry and H. Pylori

A high molecular weight, non-dialyzable material (NDM) isolated from cranberry juice inhibited the adhesion of three different strains of H. pylori to human erythrocytes and human gastric mucous (33). These bacterial strains were found to have a sialic-acid specific adhesin on their surface. It is hypothesized that the compounds from cranberry blocked the ability of this adhesin to attach to receptors on the immobilized human mucus. A follow-up study examined the adhesion behaviour of 83 strains of H. pylori, and confirmed that 0.2 mg/mL of NDM was sufficient to inhibit adhesion of 53/83 of the strains (63.86%) to gastric cells (5). This research suggested that consumption of cranberry would make it more difficult for H. pylori to colonize the mucus and the epithelium of the gut, thus representing a possible preventive measure against peptic ulcers caused by H. pylori. It may be possible to use cranberry in combination with antibiotics to prevent infections from recurring.

A randomized, double-blind, placebo-controlled clinical study investigated 189 adults infected with H. pylori (4). The cranberry juice group drank two boxes containing 150 mL cranberry juice per day for 90 days, while the control group received a placebo beverage at the same frequency and duration. At both 35 and 90 days after intervention, 14 of 97 participants (14.43%) from the cranberry group and 5 of 92 participants (5.43%) from the control group were free of H. pylori, as determined by a ¹³C-urea breath test.

### Cranberry's Action Against Oral Bacteria

Cranberry can also act against oral bacteria. For example, a high-molecular weight NDM of cranberry juice inhibited coaggregation of oral bacteria (25, 34) and reduced salivary counts of oral bacteria (34). Further, this NDM inhibited the ability of P. gingivalis to form biofilms, and prevented the microbes from attaching to surfaces coated with proteins, such as type I collagen, fibrinogen, and human serum, which represent periodontal sites (1). A pilot-type clinical study showed that six weeks of daily use of a mouthwash containing cranberry NDM reduced counts of mutans streptococci and total bacteria in saliva, compared to a control group receiving placebo mouthwash (35). Due to these encouraging results, it is likely that more clinical studies will follow.

### Cranberries and UTIs

The American red cranberry (Vaccinium macrocarpon Ait., family Ericaceae) has long been recognized for benefits to maintenance of a healthy urinary tract. This is especially a concern for women, ⅓ of whom will have at least one UTI in their lifetime (6), leading to the infection of 11.3 million women per year in the U.S. alone (7). Elderly women are also extremely prone to UTIs, with some women over 65 experiencing at least one UTI per year (8, 9).

UTIs are caused when bacteria attach to and colonize mucosa surfaces in the urinary system (10). The resulting infection can range from cystitis (bladder infection) to a more serious illness, acute pyelonephritis (kidney infection). The Gram-negative bacterium E. coli is implicated in 85-95% of cystitis and 90% of pyelonephritis infections in women (11). If untreated, UTIs can cause kidney failure, and in some cases death (10, 12, 13).

### Cranberry Effects on E. Coli—In Vivo Studies of Urinary Tract Health

The pioneering clinical trial of Avorn et al. (3) was the first to conclusively demonstrate that consumption of cranberry juice helped prevent recurrent urinary tract infections in women. This study was conducted on female residents of a long-term care facility. The women drank 300 mL/day of artificially sweetened cranberry juice or a placebo with similar color and taste for a period of 6 months. After one month, the prevalence of bacteria in the urine of the cranberry juice drinkers was significantly decreased.

Kontiokari et al. studied 150 university women (mean age of 30) who presented to the Finnish University of Oulu's student health center or occupational clinic, and had clinically documented E. coli UTIs (14). The three groups received either 1) cranberry-lingonberry juice concentrate (50 mL/day for 6 months), 2) 100 mL of a probiotic Lactobaccillus GG drink, five times per week, for one year, or 3) a control group who did not receive any intervention. The rate of recurrence of UTIs in the 12 months following the study was statistically different among these treatment groups. The overall absolute risk of recurrence of UTI was reduced by 20% for the cranberry group compared to the control group, but a benefit was not seen due to lingonberry (14).

Stothers et al. (15) studied 150 women (ages 21 to 72) who had prior histories of UTIs (≧2 in previous year), and provided them with either cranberry juice (250 mL at three times per day+placebo tablet), cranberry extract in pill form+placebo juice, or both juice and pills that were non-cranberry containing placebos, and followed the women for one year. The tablet group had the least recurrence of UTI in the following year (18%), with the cranberry juice group having a similar but significantly different recurrence rate of 20%. Both the tablet and juice groups had much lower recurrence than the non-cranberry placebo group, where 32% infection recurrence was observed.

In a pilot study of five women with culture-confirmed UTIs, participants who ate sweetened dried cranberries (SDC) in a single dose exhibited anti-adherence properties in their urine that were comparable to consuming a cranberry juice cocktail drink (16). More data from this and other clinical investigations will help demonstrate if SDCs can be used for prevention of UTIs in the same way as cranberry juice cocktail (CJC).

### Cranberry Effects on E. Coli—In Vitro Studies Related to Urinary Tract Health

While the earliest studies suggested that acidification of urine was responsible for cranberry's benefits towards UT health (17), research since the 1980s has focused on the anti-adhesive properties of cranberry juice, and recent studies demonstrated that the p1-1 of urine (after cranberry consumption) is only slightly decreased and that the effect is transient (18, 19), or showed no decrease in urine pH (2).

All uropathogenic E. coli (UPEC) isolates express protein molecules on their surfaces, known as fimbriae. These molecules include the nearly universally expressed type 1 fimbriae, which bind to a lectin on uroepithelial cells (20), and P fimbriae, which are associated with 23% of cystitis infections and nearly all pyelonephritis infections (21). Type 1 fimbriae are mannose sensitive, meaning that any mannose type sugar (i.e. fructose, common to all fruit juices) can block this protein from being able to attach to eukaryotic cells (22). P fimbriae are mannose resistant, but their binding to uroepithelial cells can be blocked by other compounds found in cranberries (23).

The ground-breaking studies demonstrating an in vivo effect of cranberry juice on bacterial adhesion to epithelial cells were performed in the 1980s, although the bacterial surface fimbriae were not investigated in these initial studies (19, 22, 24). Next, researchers began to characterize how cranberry affected bacteria with specific types of fimbriae. Zafriri et al. were the first to postulate that different compounds in cranberry could affect P and type 1 fimbriae, with their studies showing that fructose inhibited the adhesion of bacteria with type 1 fimbriae only (22). In a follow up study, these researchers tried to characterize the material that was effective against type P-fimbriated bacteria, and they determined that a high molecular weight, non-dialyzable material (NDM) inhibited the adhesion of UPEC to epithelial cells (25). A breakthrough came in 1998, when Howell et al. identified through directed fractionation, specific proanthocyanidin compounds in cranberry that caused P fimbriated-E. coli to exhibit anti-adhesion properties (23). The chemical structure of these compounds was further elucidated (26, 27). The studies of these two independent groups suggest that perhaps multiple mechanisms of anti-adhesive properties can be demonstrated against bacteria, and different compounds could be responsible for the different effects.

Current laboratory research in this area approaches the problems from multiple perspectives, including: characterization of the types of proanthocyanidins in terms of their chemical structures (28); determination of whether the beneficial compounds in cranberries are degraded by the body and elucidating their ultimate form in urine (2), microbiological studies focusing on the genes responsible for the production of fimbriae, and the role of particular fimbrial proteins in determining adhesion of the E. coli to uroepithelial cells (29), physical characterizations of the conformation and morphology of bacterial fimbriae (30), and physical interaction force measurements between E. coli bacteria and uroepithelial cells (31).

### Physical and Morphological Effects of Cranberry on E. Coli Bacteria

Bacteria were exposed to cranberry juice after growth in normal media, and we used atomic force microscopy (AFM) to probe the physical conformation of P fimbriae on E. coli HB101pDC1 that were exposed to cranberry juice cocktail (CJC) in concentrations ranging from 0 to 20% CJC (30). We found that CJC caused the P fimbriae to collapse on the surface of the E. coli cells, decreasing the protein's height and ability to extend from the surface of the bacteria. Molecular adhesion forces between the E. coli cells with collapsed fimbriae were significantly decreased compared to the molecular adhesion forces between the control (i.e. non-infective) strain of E. coli. This was the first study to quantify the molecular adhesion forces for E. coli treated with cranberry juice.

### Molecular Mechanisms of Cranberry Action Against E. Coli

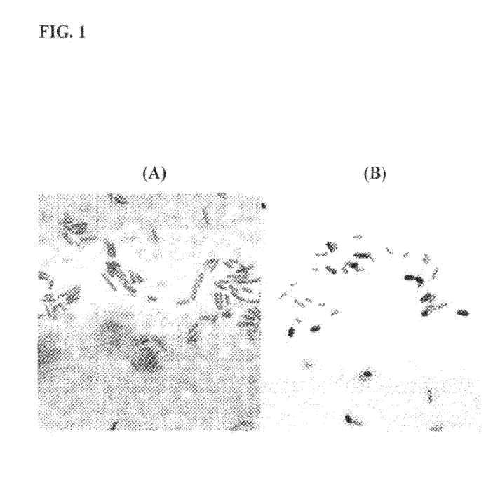

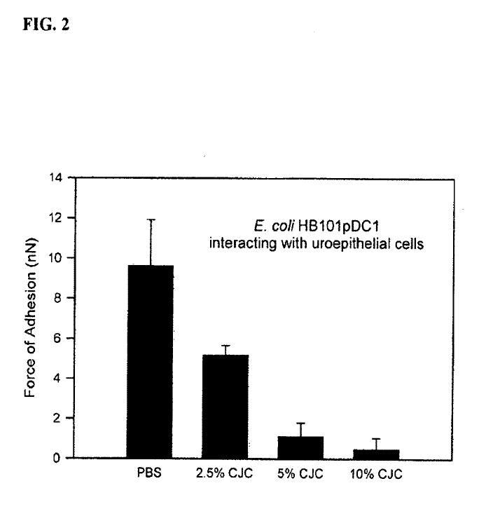

We have investigated the molecular scale effects of cranberry compounds on E. coli bacteria (31). We examined the morphology and cellular membrane properties of E. coli HB101 cells grown in culture media (tryptic soy broth; TSB) supplemented with cranberry juice, compared to E. coli grown in only TSB. The cranberry juice was neutralized to pH 7.0 before the bacterial growth experiments. The growth rate of the bacteria changed in an unpredictable manner when their growth media was supplemented with 10% CJC. Initially the bacterial growth rate decreased, but then after some time of acclimation, they resumed normal growth rates. In addition, Gram staining of the bacterial membrane revealed that culture in media supplemented with CJC changed the cellular membrane of the E. coli. For example, FIG. 1A shows E. coli HB101 bacteria grown in only TSB, and stained with a Gram stain. The E. coli appear pink, which is characteristic for Gram-negative bacteria. For the E. coli bacteria that had been grown in media supplemented with CJC, some of the cells stained pink while some stained purple (FIG. 1B). The purple appearance is an indication of Gram-positive bacteria and is an unusual finding for E. coli. While the mechanism of action is not yet clear, we speculate that some compounds from the cranberry juice are altering either the peptidoglycan layer or lipopolysaccharide layer of the E. coli, causing these apparent changes in the cell wall organization.

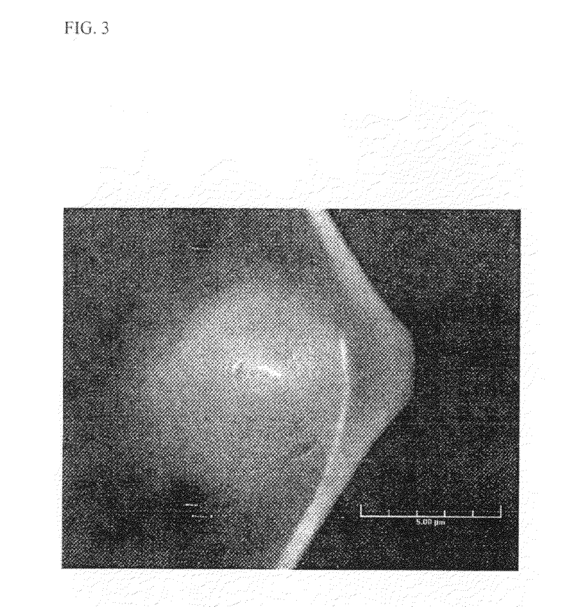

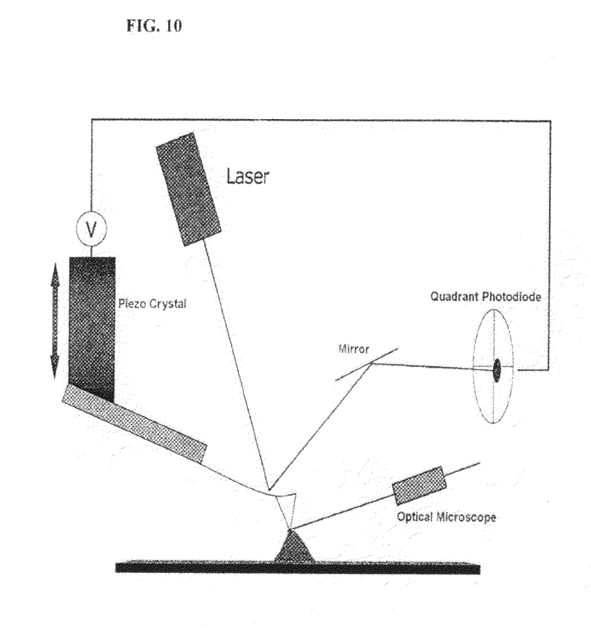

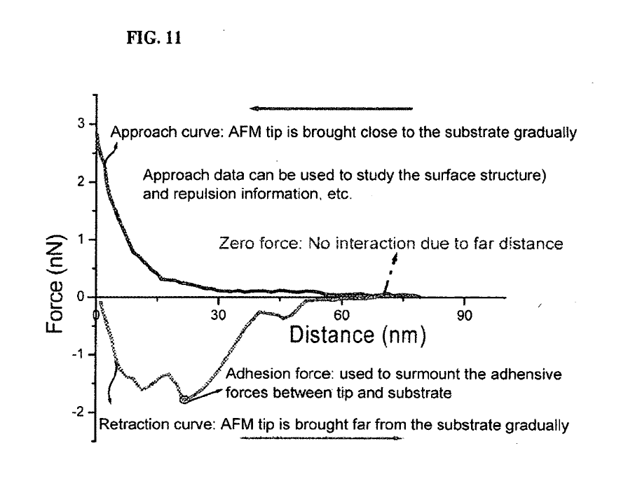

The current invention involves the use of a nanotechnology-based tool, atomic force microscopy (AFM) (see FIG. 10), to measure the nanoscale adhesion forces between E. coli bacteria and uroepithelial cells. By combining nanoscopic force measurements with calculations of the interaction energies surrounding bacteria and uroepithelial cells, we have found that cranberry juice affects the nature of the E. coli-uroepithelial cell in several ways: 1) cranberry juice causes P fimbriae on the E. coli to collapse, thus being unable to form attachments to uroepithelial cells (30), 2) cranberry juice causes an “energy barrier” to build up around the E. coli and the uroepithelial cells, thus making it unfavourable for the two to make contact with one another (31), and 3) cranberry juice decreases the forces of adhesion between P fimbriated E. coli and urinary tract cells from 9.64 nN (in buffer alone) to 0.50 nN (in buffer plus 10% cranberry juice; FIG. 2) (31). Our nanoscale measurements can help to elucidate the mechanisms by which cranberry compounds can block the adhesion of E. coli bacteria to uroepithelial cells.

The current invention involves a novel method to functionalize the AFM tip that allows for single E. coli bacteria to be reproducibly attached to the top of the AFM tip. This functionalized AFM tip gives reproducible and comparable force measurements and yields accurate values when coupled with statistical analysis. The current invention makes use of living cells (bacteria and epithelial cells) as the two surfaces of interest. Previous methods only considered one component of the system interfacial free energy, i.e. the surface free energy of the substrate and ignored the other two components, those arising from the bacterial cells and the medium.

When considering the very small magnitude of interaction forces involved in cell-cell interactions or cell-matrix interactions, a well functionalized AFM tip is crucial to detect those subtle forces. Such functionalization must preserve the true nature of the cell surface. In our invention, since we use intact epithelial cells and intact bacterial cells, the ligand and receptor are already in their correct orientation.

Bacterial cells and uroepithelial cells are treated with the solution of desired compounds, i.e. the component(s) solution with a certain concentration for a certain time. For the AFM-based bioassay, bacterial cells are attached to the AFM tip to make sure only one bacterium is located on the top of the AFM tip. The bacteria-functionalized AFM tip is used to probe the uroepithelial cells in the same solution that is used to treat the cells. AFM force cycles of both approach curves and retraction curves are collected. Then adhesion forces are analyzed and statistical analysis is utilized to immediately tell the effects of this component, or the concentration or the time of exposure.

For interfacial free energy analysis, bacterial cells and uroepithelial cells are placed on 0.45 and 0.8 micron isopore filter membranes, with the help of filtration after the component(s) treatment. Then three different probe solutions, namely water, diiodomethane and formamide, are used to measure the corresponding contact angles. Based on those contact angles, surface free energy of individual substrata is calculated. The interfacial free energy of each two interface system is derived from the individual surface free energies. Then the system interfacial free energy is calculated from the three sets of two-interface interfacial free energies. After different formulations of compounds from cranberry juice are screened, detailed mechanistic studies and characterizations can be carried out. Also, clinical studies can make more informed choices of the dose and time needed for best response.

Investigations were conducted to examine the molecular-scale effects of cranberry juice on adhesion between mammalian cells and bacterial dells. More specifically, nanoscale tool, atomic force microscopy (AFM) was used to investigate bacterial surface characteristics and directly measure the strength of the adhesion forces between individual E. coli bacteria and uroepithelial cells. Two strains of E. coli: HB101, which has no fimbriae, and the mutant strain HB101pDC1, which expresses P-fimbriae and is responsible for acute pyelonephritis were attached to the AFM tip. The uroepithelial cells, SV-HUC-1, human kidney cells (ATCC CRL-9520) were used. Note that the uroepithelial cells here include the epithelial cells coming from the urinary tract, namely the kidneys, the ureters (the tubes that take urine from each kidney to the bladder), the bladder, or the urethra (the tube that empties urine from the bladder to the outside). The uropathogenic bacteria include Escherichia coli, Chlamydia and Mycoplasma.

Cranberries and their associated juice and food products represent a multi-billion dollar industry. Our findings related to the anti-adhesive benefits of cranberry juice can advance the commercial viability of products that already exist and are important parts of the economy. In addition to the juice/food products based on cranberries, dietary supplements are also being prepared based on cranberries or the extracted proanthocyanidins. A challenge in this industry is that it can be difficult to know the bioactivity associated with mixtures of natural products. Our bioassay can be used to determine precisely how much of a particular dietary supplement is needed in order to have actions against E. coli that will benefit urinary tract health. The activity of these substances can be made more effective and reliable based on our research findings.

Cranberry has long been known to benefit the urinary tract health, however the detailed mechanisms to describe how it is beneficial are still missing. Recently, cranberry was also recognized to act against the bacteria that cause gastric ulcers and periodontitis, suggesting that cranberry ingestion can prevent these other illnesses, as well. Escherichia coli bacteria are the predominant uropathogen causing UTIs. Helicobacter pylori bacteria are the culprit leading to gastric ulcers. Gram-negative bacteria such as Filifactor alocis, Streptococcus mutans, and Treponema socranskii are responsible for periodontitis. Although each system is different, the reasons cranberry causes benefits are believed to be the same, i.e. through interference of the ability of the bacteria to bind to human tissue, proteins, or other cells. The established methodology in our invention can be applied towards these other infections to obtain similar goals.

Because the beneficial compounds (proanthocyanidins and/or non-dialyzable material) represent families of materials, a huge challenge remains in determining how to accurately and efficiently quantify the potency of a particular cranberry product. Our assay allows for the determination of the amount of effectiveness a cranberry product has against bacterial adhesion to uroepithelial cells.

### Examples

### Bacterial Cells Culture

Bacterial cells such as Escherichia coli and Staphylococcus were precultured in appropriate medium such as Tryptic Soy Broth (TSB) overnight at required temperature such as 37° C. Then bacteria from the preculture were cultured to designed growth stage such as middle exponential phase at required temperature such as 37° C. The growth stage was monitored by measuring the absorbance with the help of a spectrophotometer at 600 nm wavelength. To measure the absorbance, pure culture medium such as TSB solution was used to set the blank value.

Bacterial cells were harvested in the middle exponential phase and then were collected by centrifugation for 5˜15 min at 1000˜2000 g. The supernatant was decanted and then the bacterial cells were washed with PBS (pH 7.4, NaCl 0.138 M, KCl 0.0027 M, K₂HPO₄ 0.005 M, KH₂PO₄ 0.005 M). Next, the bacteria solution was centrifuged to remove the solution. The above process was taken as the first washing step. Three washing steps were employed to fully remove the components of the growth medium retained-stuck on the bacterial surface after growth; Gallardo-Moreno, A. M., Liu, Y., González-Martín, M. L. & Camesano, T. A. Atomic Force Microscopy Analysis of Bacterial Surface Morphology Before and After Cell Washing. Journal of Scanning Probe Microscopy 1, 63-73 (2006), the entire contents of which are incorporated herein by reference.

### Mammalian Cells Culture

Mammalian cells such as epithelial cells were kept in liquid nitrogen vapor phase as long term storage. The cells were grown in required medium such as Kaighn's modification of Ham's F12 medium and supplemented with 10% fetal bovine serum. Tissue culture flasks were kept in a desired CO₂ concentration such as 5˜10% in air atmosphere incubator at 37° C. for 6-7 days where the media was replaced every other day. The cells were harvested by adding 0.25% (w/v) Trypsin-0.03% (w/v) EDTA to detach the cells from the culture flask. After centrifugation the cells were resuspended in the desired concentration of cranberry juice media for three hours.

### Cranberry Juice Treatment

Here we refer commercial available cranberry juice cocktail, concentrated cranberry juice or certain cranberry compound(s) solution such as proanthocyanidins as “cranberry juice” hereafter in general. Prior to use, cranberry juice was neutralized to exclude the effects of low pH. A series of concentration solutions, 2.5˜50.0 wt. % cranberry juice, were prepared from the original cranberry juice by adding 0.01 M phosphate buffered saline (PBS) solution (NaCl 0.138 M; KCl 0.0027 M) at pH=7.4.

Bacterial cells and mammalian cells were immersed in 0.01 M PBS, cranberry juice solutions of different concentration for 3 hours at 70 rpm rotation at 37° C. respectively.

### Contact Angles Measurement

The contact angles of ultrapure water, diiodomethane and formamide were measured using the sessile drop technique (see, Busscher, H. J. et al. Measurement of the Surface Free Energy of Bacterial Cell Surfaces and Its Relevance for Adhesion. Applied And Environmental Microbiology 45, 980-983 (1984); the entire contents of which are incorporated herein by reference) with the help of a goniometer at designed temperature and ambient humidity.

Bacterial cells (3˜9×10⁹ cells) and uroepithelial cells (5˜10×10⁶ cells) were deposited on 0.45 μm and 8 μm pore size cellulose acetate filters respectively via suction filtration to form cell lawns. After freshly deposition, a certain drying time is required to evaporate the excess moisture among the cells. The drying time can be determined by measuring the water contact angles as a function of time. After a time threshold, water contact angles reached a plateau. Under this situation, only the moisture retained by the cell surface structures remained, which was the right state used for three liquid contact angle measurements; van Oss, C. J. Interfacial Forces in Aqueous Media. (Marcel Dekker, Inc., New York, N.Y.; 1994), the entire contents of which arc incorporated herein by reference.

At least 3 replicates of probe liquid contact angles were measured per filter and at least 4 filters were analyzed for each condition. Contact angles were based on at least 12 measurements per condition.

### Surface Free Energy of Individual Substrata and System Interfacial Free Energy Change Calculations

The contact angle measurements still remain as the most accurate force balance methodology for quantifying the interactions between two liquid and solid at the minimum equilibrium distance, i.e. at molecule contact; van Oss, C. J. Interfacial Forces in Aqueous Media. (Marcel Dekker, Inc., New York, N.Y.; 1994), the entire contents of which are incorporated herein by reference. The surface free energy, γ, is a summary of two terms, Lifshitz-van der Waals (LW) surface free energy component γ^{LW} and the acid-base (AB) surface free energy component γ^{AB}, which is the geometric mean of two components, i.e. the electron-donor (γ⁻) and electron-acceptor (γ⁺) parameters. Hence, the surface free energy can be expressed as:

γ=Γ^{LW}+2·\sqrt{γ⁺·γ⁻} (1)

When a drop of a liquid (L) is deposited on a solid surface (S), the contact angle between the drop and the surface (θ) is a function of the components and parameters of the surface free energy of the liquid and the solid. The Young-Dupré equation relates such magnitudes:

γ_{L}(cos θ_{L}+1)=2·\sqrt{γ_{S}^{LW}·γ_{L}^{LW}}+2·\sqrt{γ_{S}⁺·γ_{L}⁻}+2·\sqrt{γ_{S}⁻·γ_{L}⁺} (2)

If γ_{L}^{LW}, γ_{L}⁻ and γ_{L}⁺ are known, then γ_{S}^{LW}, γ_{S}⁻ and γ_{S}⁺ can be calculated.

Three equations are required to solve these three unknowns, which is the reason of using three probe liquids with different polarity for contact angle measurements.

γ_{W}(cos θ_{W}+1)=2·\sqrt{γ_{S}^{LW}·γ_{W}^{LW}}+2·\sqrt{γ_{S}⁺·γ_{W}⁻}+2·\sqrt{γ_{S}⁻·γ_{W}⁺} (3)

γ_{D}(cos θ_{D}+1)=2·\sqrt{γ_{S}^{LW}·γ_{D}^{LW}}+2·\sqrt{γ_{S}⁺·γ_{D}⁻}+2·\sqrt{γ_{S}⁻·γ_{D}⁺} (4)

γ_{F}(cos θ_{F}+1)=2·\sqrt{γ_{S}^{LW}·γ_{F}^{LW}}+2·\sqrt{γ_{S}⁺·γ_{F}⁻}+2·\sqrt{γ_{S}⁻·γ_{F}⁺} (5)

W, D, F denoting water, diiodomethane, and formamide, respectively.

By solving equations 3-5 simultaneously, we obtain:

$$[ \begin{matrix}{γ}_{S}^{LW} \\ {γ}_{S}^{+} \\ {γ}_{S}^{-}\end{matrix} ] = {\{ \begin{matrix}{[ 2 \cdot ( \begin{matrix}\sqrt{{γ}_{W}^{LW}} & \sqrt{{γ}_{W}^{-}} & \sqrt{{γ}_{W}^{+}} \\ \sqrt{{γ}_{D}^{LW}} & \sqrt{{γ}_{D}^{-}} & \sqrt{{γ}_{D}^{+}} \\ \sqrt{{γ}_{F}^{LW}} & \sqrt{{γ}_{F}^{-}} & \sqrt{{γ}_{F}^{+}}\end{matrix} ) ]}^{- 1} \cdot \\ ( \begin{matrix}{γ}_{W} \cdot [ cos ( {θ}_{W} ) + 1 ] \\ {γ}_{D} \cdot [ cos ( {θ}_{D} ) + 1 ] \\ {γ}_{F} \cdot [ cos ( {θ}_{F} ) + 1 ]\end{matrix} )\end{matrix} \}}^{2} \qquad ( 6 )$$

These calculations can be applied to the obtaining of the components and parameters of the surface free energy of bacterial or uroepithelial cells (S) immersed in the different conditions of this study.

If bacterial cells can adhere onto uroepithelial cells, a new interface (bacterium-uroepithelial cell: B-UC) will form at the expanse of losing two old interfaces (bacterium-suspending liquid: B-L and uroepithelial cell-suspending liquid: UC-L). The system interfacial free energy change ΔG_{adh} is the difference between the final state and initial state (van Oss, C. J. Interfacial Forces in Aqueous Media. (Marcel Dekker, Inc., New York, N.Y.; 1994), the entire contents of which are incorporated herein by reference):

ΔG_{adh}=γ_{B-UC}−γ_{B-L}−γ_{UC-L} (7)

where, γ_{B-UC}, γ_{B-L} and γ_{UC-L} denote the interfacial free energy (mJ·m⁻²) of the interfaces bacterium-uroepithelial cell, bacterium-suspending liquid and uroepithelial cell-suspending liquid, respectively.

The interfacial free energy between subject 1 and subject 2 (i.e. γ_{B-UC}, γ_{B-L} and γ_{UC-L}) can be calculated as:

γ₁₂=(\sqrt{γ₁^{LW}}−\sqrt{γ₂^{LW}})²+2·[(\sqrt{γ₁⁺}−\sqrt{γ₂⁺})·(\sqrt{γ₁⁻}−\sqrt{γ₂⁻})] (8)

Similarly to the division of the surface free energy into two components, also the system interaction free energy change immersed in water (W) can be calculated as the sum of system LW interfacial free energy change and AB interfacial free energy change:

ΔG_{adh}=ΔG_{adh}^{LW}+ΔG_{adh}^{AB} (9)

where

ΔG_{adh}^{LW}=(\sqrt{γ_{B}^{LW}}−\sqrt{γ_{UC}^{LW}})²−(\sqrt{γ_{B}^{LW}}−\sqrt{γ_{W}^{LW}})²−(\sqrt{γ_{UC}^{LW}}−\sqrt{γ_{W}^{LW}})² (10)

ΔG_{adh}^{AB}=2·[\sqrt{γ_{W}⁺}·(\sqrt{γ_{B}⁻}+\sqrt{γ_{UC}⁻}−\sqrt{γ_{W}⁻})+\sqrt{γ_{W}⁻}·(\sqrt{γ_{B}⁺}+\sqrt{γ_{UC}⁺}−\sqrt{γ_{W}⁺})−\sqrt{γ_{B}⁺·γ_{UC}⁻}−\sqrt{γ_{B}⁻·γ_{UC}⁺}] (11)

### Bacteria Adherence Assay

Bacteria, prepared as aforementioned protocol, were immersed in an aqueous solution of different cranberry juice concentration for a period of 3 hours at desired temperature. At the same time, harvested uroepithelial cells were also immersed in aqueous solutions that contained the same concentration of cranberry juice.

After cranberry juice treatment bacteria at a concentration of 10⁹ cells/mL and uroepithelial cells (10⁶ cells/mL) were incubated in tissue culture flasks at 37° C. for 90 minutes at a speed of 18 rpm. Wet mounts were then prepared and the number of bacteria adhered to at least 20 uroepithelial cells was determined for each condition. A microscope with a 1000 magnification was used with an oil immersion 100×, 1.33 numerical aperture objective. The mammalian cells were observed under phase contrast microscopy using a DIA ILL (A) filter block. The images were taken and the adhered bacteria were counted at least on 20 epithelial cells for each condition.

### Coating One Bacterium Onto an AFM Tip

### AFM Tip Preparation

Commercially available AFM tips were utilized, including silicon, silicon nitride and gold-coated tips. The AFM cantilevers comprise rectangular or triangular shapes. The curvature of the AFM tips are preferred to be around 10˜50 nm. Prior to use, the AFM tips were exposed to the UV light for 10˜30 minutes to remove any organic contaminations.

Positive polymer molecules such as poly-L-Lysine, poly-(ethyleneimide) with certain concentration ranges were spread onto flat hydrophilic surfaces to form a thin liquid film. The AFM tips were mounted onto in air tip holder. Align the laser and set the control parameters such as setpoint, proportional gain and integral gain to appropriate values. Prior to the engagement, the scan size was decreased to 1˜10 nm and the scan rate was decreased to 0.1˜0.5 Hz. Then the AFM tips were engaged onto the thin liquid film either in contact mode or tapping mode via careful adjustment. The AFM tips were allowed to scan the thin liquid film for 30 seconds to 3 minutes. Then the AFM tips were carefully retracted and kept in the closed clean AFM chamber to eliminate the contact with the dusts in the air.

### Bacterial Cells Preparation

Bacteria were cultured and washed according to the aforementioned protocol. After three times' washing, bacterial cells were spin down by centrifugation force at around 1000˜2000 g or small filtration forces. Then the cell pellet was placed onto hydrophobic surfaces to form a 100˜10000 cell layer bacteria film. The positive polymer molecules functionalized AFM tips were used to probe the bacteria film Similar to the above engagement, the AFM operation parameters were adjusted before engagement. The scan size was decreased to 1˜10 nm and the scan rate was decreased to 0.1˜0.5 Hz. Then the optical microscope was used to locate the engagement location. Firstly, the boundary of bacteria film and clean substrate was located. Secondly, the AFM tip was adjusted above the bacteria film just across the boundary and the most part of the AFM cantilever was above the clean substrate. Thirdly, the distance between the AFM tip and the bacteria film should be well adjusted to avoid failed engagement due to short distance. The AFM tip was successfully engaged onto the bacteria film in either contact or tapping mode. The AFM tip was allowed to scan the surface for 30 seconds to 1 minute. The AFM tip was carefully retracted and kept in air tip holder for around ¼˜⅓ of the bacteria drying time determined via water contact angle experiments. Then the bacterium-functionalized AFM tip was transferred to liquid AFM tip holder to perform following force measurements.

Here, the electrostatic forces together with the mechanical forces exerted by AFM are the driving force to immobilize bacteria onto the AFM tip. The sensitive AFM control loop, the sharp AFM tip and the nano-scale contact between AFM tip and bacteria allow single bacterium immobilized onto the AFM tip.

### Verification of Successful Functionalization

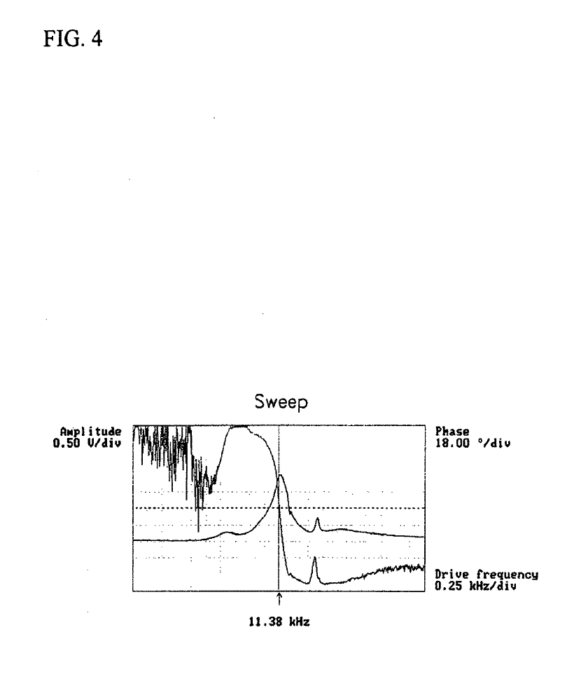

There are three methods to verify the successful bacteria functionalization:
(1) Scanning electron microscope (SEM) imaging. The functionalized AFM tip before or after force measurement can be imaged by SEM (see FIG. 3);
(2) Resonance frequency shift The AFM can auto tune the resonance frequency of the mounted AFM tip as shown in FIG. 4. The resonance frequency is a function of the effective mass of the AFM cantilever as given in the following equations.

$${f}_{1} = \frac{1}{2 π} \sqrt{\frac{k}{M}}$$

$${f}_{2} = \frac{1}{2 π} \sqrt{\frac{k}{M + Δ {m}_{C}}}$$

$${f}_{3} = \frac{1}{2 π} \sqrt{\frac{k}{M + Δ {m}_{C} + Δ {m}_{B}}}$$

where
Δm_{C}: the mass of added chemical molecules
Δm_{B}: the coated bacteria mass
f₁, f₂, f₃: resonance frequency of the cantilever of the bare tip, chemically modified tip and bacteria coated tip, respectively

Then, we have:

$$Δ {m}_{B} = \frac{k}{4 {π}^{2}} \cdot ( \frac{1}{{f}_{3}^{2}} - \frac{1}{{f}_{2}^{2}} )$$

Based on the normally used AFM tip spring constants calibrated in our lab, the sensitivity analysis can give the lower boundary of mass detection,

$$\frac{Δ {m}_{B}}{Δ f} = \frac{k \cdot {10}^{12} \cdot 1000}{4 \cdot {π}^{2}} \cdot \frac{{f}_{2} + {f}_{3}}{{f}_{2}^{2} \cdot {f}_{3}^{2}} = 0.707 ∼ 7.07 pg \text{/} KHz$$

The mass resolution is extremely high, which also shows a light for AFM to he used as a sensitive bio-sensor.

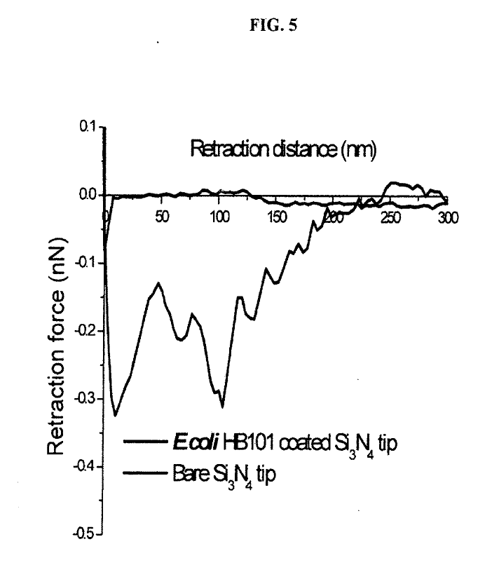

After positive polymer coating or bacteria coating, the resonance frequency shifts can be recorded to quantify the amount of coated polymer molecules or bacteria mass.
(3) Characteristic force spectra. The difference in the characteristic force spectra can distinguish bacteria coated tip or uncoated tip, which can be used to verify the successful functionalization immediately. The bare AFM tip is relatively rigid. Thus the interaction forces between bare tip and bare surface are usually short distance and only have one retraction peak as shown in a sample in FIG. 5.

When conducting the bacterium-functionalized AFM force measurement, before and after force measurement, the forces on the bare surface such as glass were always checked to verify the successful functionalization.

### Interaction Forces Between Bacteria and Epithelial Cells

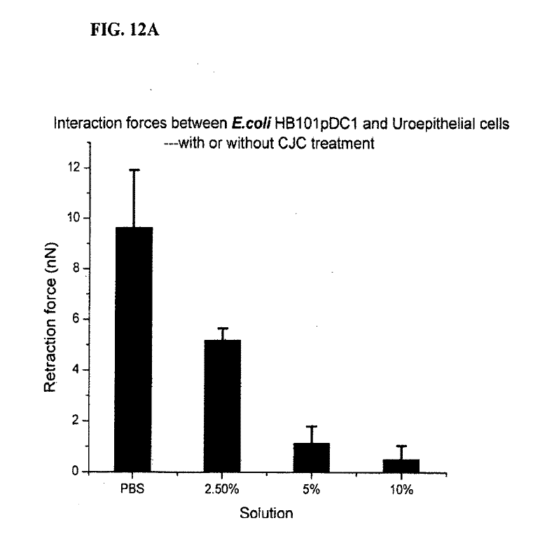

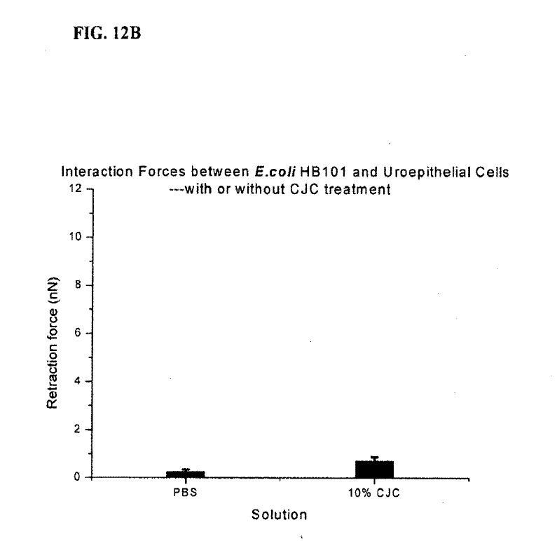

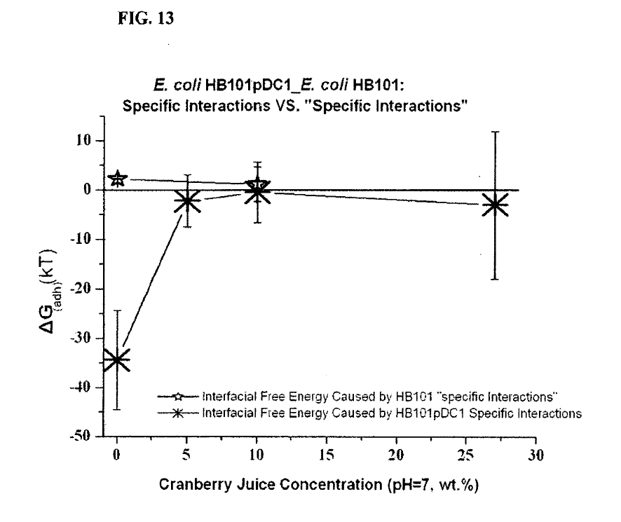

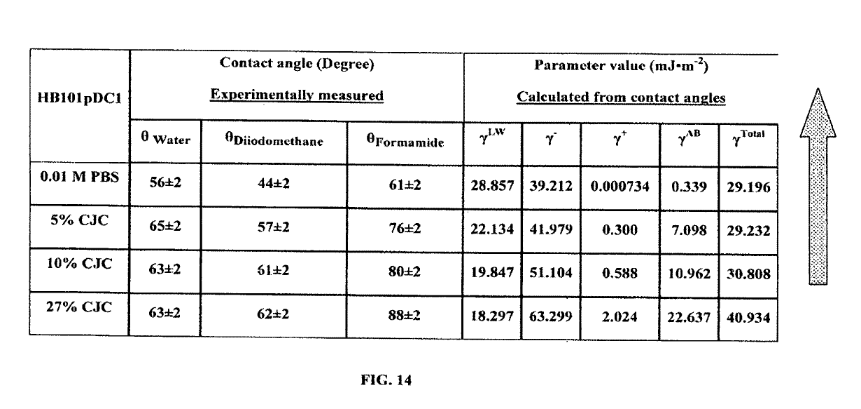

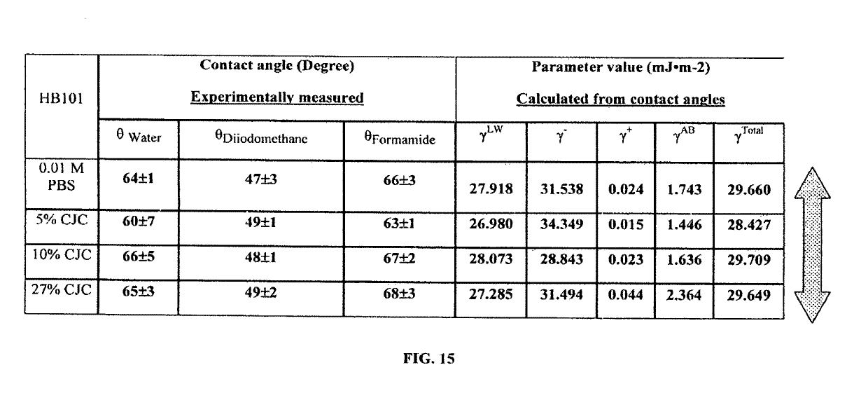

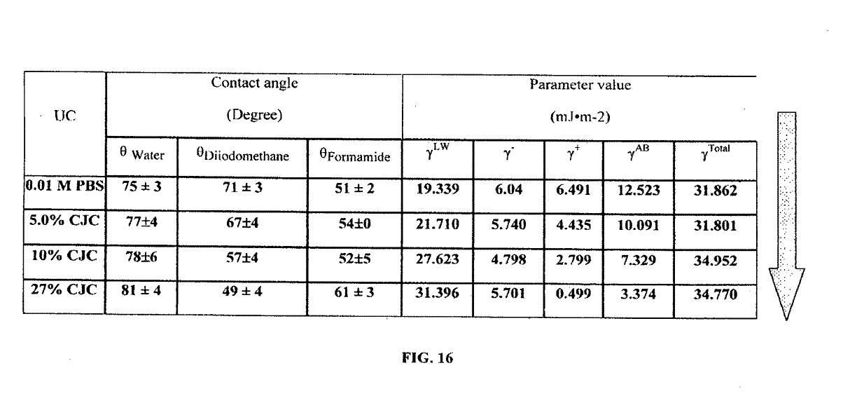

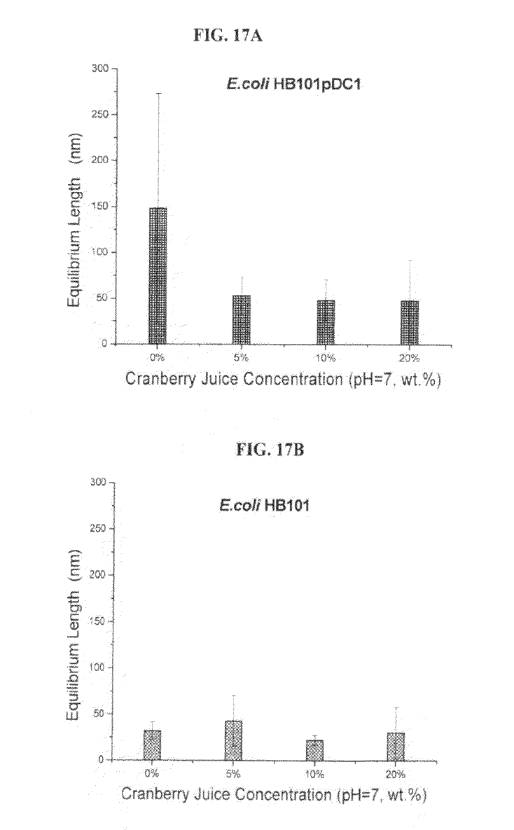

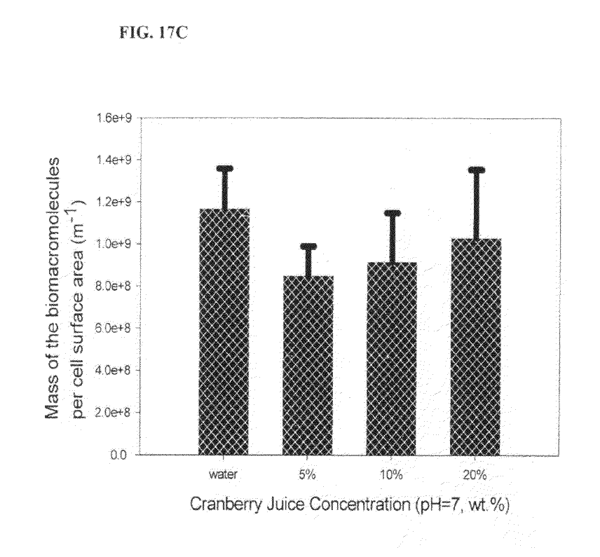

The adhesion forces between E. coli HB101pDC1 and the uroepithelial cells decreased when the concentration of CJC was increased. Also a concentration threshold was found at between 2.5-5.0 wt. %, with lower CJC concentrations than the threshold unable to cause a reduction in the adhesion force. Also, the adhesion forces between E. coli HB101 and the uroepithelial cells are much smaller than that of the P-fimbriated bacteria. See FIGS. 11-13.

### Surface Free Energy Investigation

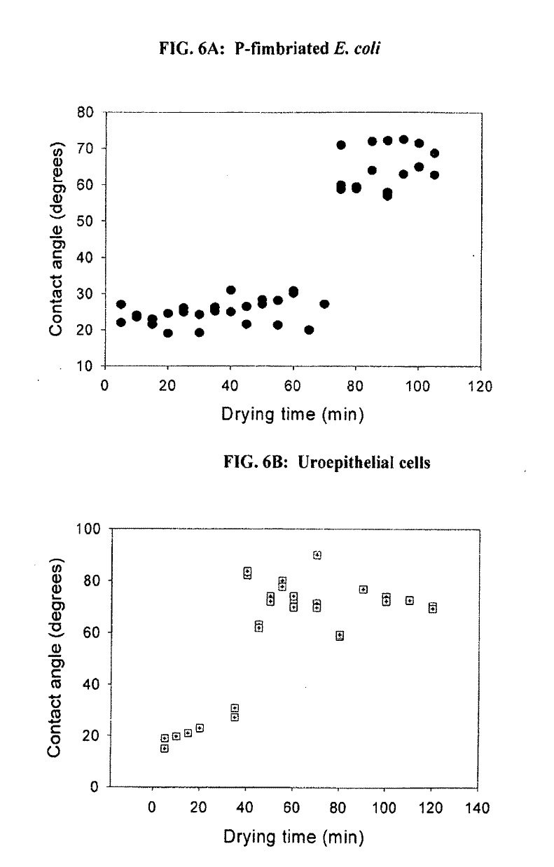

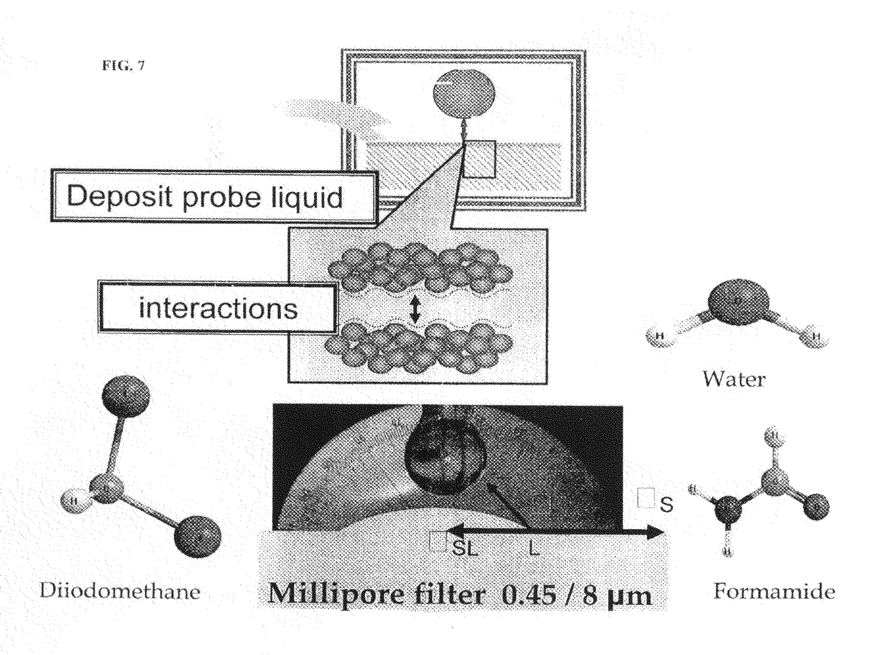

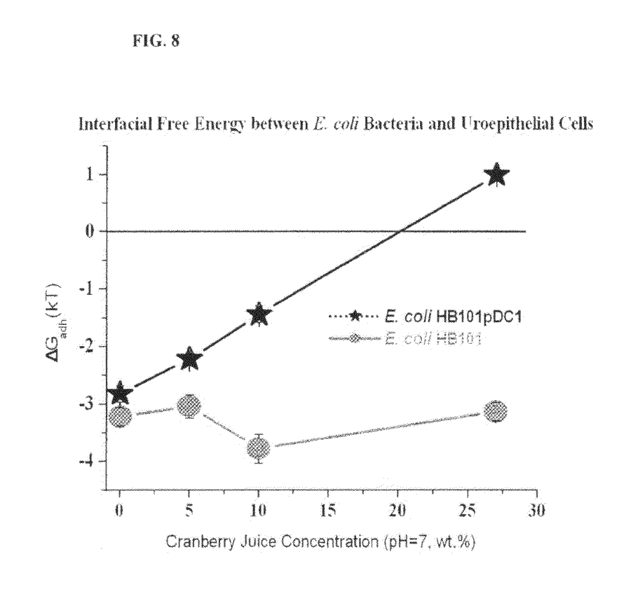

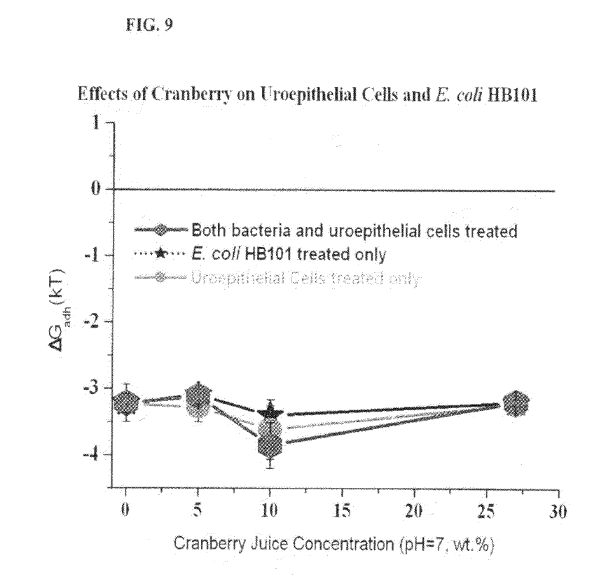

Both for E. coli HB101pDC1 and uroepithelial cells, the contact angles experience a large change after a certain time, which represents the drying time for that type of cell (FIGS. 6A and 6B).

For CJC concentrations >2.5%, cranberry alters the cell surfaces by making them more hydrophobic. The total surface free energy is nearly the same after the treatment with CJC, but the degree of the asymmetry between the electron acceptor and electron donor terms implies that CJC has made the cell surfaces less hydrophilic. The effect will occur when the exposure time is around three hours. A similar time of exposure to CJC was needed to cause changes in the P-fimbriae morphology and to decrease the adhesion forces.

### REFERENCES

1. Labrecque, J., C. Bodet, F. Chandad, and D. Grenier, Journal of Antimicrobial Chemotherapy, 58, 439-443, (2006).
2. DiMartino, P., R. Agniel, K. David, C. Templer, J. L. Gaillard, P. Denys, and H. Botto, World Journal of Urology, 24, 21-27, (2006).
3. Avorn, J., M. Monane, J. H. Gurwitz, R. J. Glynn, I. Choodnovskiy, and L. A. Lipsitz, Journal of the American Medical Association, 271, 751-754. (1994).
4. Zhang, L., J. Ma, K. Pan, V. L. W. Go, J. Chen, and W.-C. You, Helicobacter, 10, 139-145, (2005).
5. Shmuely, H., O. Burger, I. Neeman, J. Yahav, Z. Samra, Y. Niv, N. Sharon, E. Weiss, A. Athamna, M. Tabak, and I. Ofek, Diagnostic Microbiology and Infectious Disease, 50, 231-235, (2004).
6. Sobel, J. D., Medical Clinics of North America. 253-273, (1991).
7. Foxman, B., R. Barlow, H. D'Arcy, B. Gillespie, and J. D. Sobel. Annals of Epidemiology, 10, 509-515, (2000).
8. Boscia, J. A., W. D. Kobasa, R. A. Knight, E. Abrutyn, M. E. Levison, and D. Kaye, American Journal of Medicine, 80, 208-214, (1986).
9. Abrutyn, E., J. Mossey, M. Levison, J. Boscia, P. Pitsakis, and D. Kaye, Journal of the American Geriatric Society, 39, 388-393, (1991).
10. Karkkainen, U. M., J. Kauppinen, R. Ikaheimo, M. L. Katila, and A. Siitonen, Journal of Microbiological Methods, 34, 23-29, (1998).
11. Russo, T. A. and J. R. Johnson, Microbes Infection, 5, 449-456, (2003).
12. Faro, S. and D. E. Fenner, Clin Obstet Gynecol, 41, 744-54, (1998).
13. Bahrani-Mougeot, F. K., E. L. Buckles, C. V. Lockatell, J. R. Hebei., D. E. Johnson, C. M. Tang, and M. S. Donnenberg, Molecular Microbiology, 45, 1079-1093, (2002).
14. Kontiokari, T., K. Sundqvist, M. Nuutimen, T. Pokka, M. Koskela, and M. Uhari, BMJ, 322, (2001).
15. Stothers, L. A., Canadian Journal of Urology, 9, 1558-1562, (2002).
16. Greenberg, J. A., S. J. Newmann, and A. B. Howell, The Journal of Alternative and Complementary Medicine, 11, 875-878, (2005).
17. Blatherwick, N. R., Archives of Internal Medicine, 14, 409-450, (1914).
18. Walsh, B. A., Journal of ET nursing: official publication, International Association for Enterostomal Therapy, 19, 110-3, (1992).
19. Sobota, A. E., Journal of Urology, 131, 1013-1016, (1984).
20. Emody, L., M. Kerenyi, and G. Nagy, International Journal of Antimicrobial Agents, 22, 29-33, (2003).
21. Arthur, M., C. E. Johnson, R. H. Rubin, R. D. Arbeit, C. Campanelli, C. Kim, S. Steinbach, M. Agarwal, R. Wilkinson, and R. Goldstein, Infection and Immunity, 57, 303-313, (1989).
22. Zafriri, D., I. Ofek, R. Adar, M. Pocino, and N. Sharon, Antimicrobial Agents and Chemotherapy, 33, 92-98, (1989).
23. Howell, A. B., N. Vorsa, A. D. Marderosian, and L. Y. Foo, New England Journal of Medicine, 339, 1408-1408, (1998).
24. Schmidt, D. R. and A. Sobota, Microbios, 55, 173-181, (1988).
25. Ofek, L. J. Goldhar, D. Zafriri, H. Lis, R. Adar, and N. Sharon, New England Journal of Medicine, 324, 1599, (1991).
26. Foo, L. Y., Y. R. Lu, A. B. Howell, and N. Vorsa, Phytochemistry, 54, 173-181, (2000).
27. Foo, L. Y., Y. R. Lu, A. B. Howell, and N. Vorsa, Journal of Natural Products, 63, 1225-1228, (2000).
28. Howell, A. B., J. D. Reed, C. G. Krueger, R. Winterbottom, D. G. Cunningham, and M. Leahy, Phytochemistry, 66, 2281-2291, (2005).
29. Mulvey. M. A., Cellular Microbiology. 4. 257-271. (2002).
30. Liu, Y., M. A. Black, L. Caron, and T. A. Camesano, Biotechnology and Bioengineering, 93, 297-305, (2006).
31. Liu, Y., A. M. Gallardo-Moreno, and T. A. Camesano. Effects of cranberry juice on the physicochemical properties of Escherichia coli and uroepithelial cells. in American Chemical Society 232nd National Meeting. 2006. San Francisco, Calif.
32. Ahuja, S., M. B. Kaack, and J. A. Roberts, Journal of Urology, 159, 559-562, (1998).
33. Burger, O., I. Ofek, M. Tabak, E. I. Weiss. N. Sharon, and I. Neeman, FEMS Immunology and Medical Microbiology, 29, 295-301, (2000).
34. Weiss, E. L., R. Lev-Dor, N. Sharon, and I. Ofek, Critical Reviews in Food Science and Nutrition, 42(Suppl), 285-292, (2002).
35. Weiss, E. I., A. Kozlovsky, D. Steinberg, R. Lev-Dor, R. B. N. Greenstein, M. Feldman, N. Sharon, and I. Ofek, FEMS Microbiology Letters, 232, 89-92, (2004).

What is claimed is:

1. A method for functionalizing an atomic force microscope tip comprising attaching a single cell to the top of said tip.

2. The method of claim 1, wherein attaching comprises
coating said tip with positively charged polymer molecules;
contacting said polymer coated tip to a cell suspension; and
verifying the immobilization of a cell onto said tip.

3. The method of claim 2, wherein said tip is selected from the group consisting of silicon tips, silicon nitride tips and gold-coated tips.

4. The method of claim 2, wherein said positively charged polymer molecule is poly-L-Lysine or poly-(ethyleneimide).

5. The method of claim 2, wherein said cell suspension is a film of bacteria.

6. The method of claim 2, wherein said verifying step is a method selected from the group consisting of imaging by scanning electron microscopy, determining the resonance frequency shift, and measuring the difference in the characteristic force spectra.

7. The method of claim 1, wherein said single cell is a bacteria cell.

8. The method of claim 7, wherein said bacteria cell is selected from the group consisting of Escherichia coli, Helicobacter pylori and Porphyromonas gingivalis.

9. A method for measuring adhesion forces between two cells by determining the system interfacial free energy comprising:
measuring the surface free energy of a first cell;
measuring the surface free energy of a second cell; and
measuring the surface free energy of a medium.

10. The method of claim 9, wherein said lirst cell is a bacteria cell.

11. The method of claim 10, wherein said bacteria cell is selected from the group consisting of Escherichia coli, Helicobacter pylori and Porphyromonas gingivalis.

12. The method of claim 9, wherein said second cell is a mammalian cell.

13. The method of claim 12, wherein said mammalian cell is selected from the group consisting of epithelial cells, erythrocytes.

14. The method of claim 9, wherein said medium is comprised of between about 2.5 wt. % and 30 wt. % cranberry juice.

15. A method for measuring adhesion forces between a cell and a substrate by determining the system interfacial free energy comprising:
measuring the surface free energy of said cell;
measuring the surface free energy of said substrate; and
measuring the surface free energy of a medium.

16. The method of claim 15, wherein said first cell is a bacteria cell.

17. The method of claim 16, wherein said bacteria cell is selected from the group consisting of Escherichia coli, Helicobacter pylori and Porphyromonas gingivalis.

18. The method of claim 15, wherein said substrate is gastric mucous.

19. The method of claim 15, wherein said substrate is a surface coated with protein.

20. The method of claim 19, wherein said protein is selected from the group consisting of type 1 collagen, fibrinogen, and serum and said surface is periodontal tissue or teeth.

21. The method of claim 15, wherein said medium is comprised of between about 2.5 wt. % and 30 wt. % cranberry juice.

22. A method of screening for compounds that disrupt cell-cell interactions comprising:
incubating said cells with a compound or mixture of compounds; and
measuring the adhesion forces between said cells according to the method of claim 9.

23. A method of screening for compounds that disrupt cell-substrate interactions comprising:
incubating said cells and substrates with a compound or mixture of compounds; and
measuring the adhesion forces between said cells and substrate according to the method of claim 15.
